(19) United States
(12) Patent Application Publication
(10) Pub. No.: US 2026/0215701 A1
(43) Pub. Date: Jul. 30, 2026

(54) MICRO-DOPPLER RADAR SYSTEMS AND METHODS FOR EVALUATING HUMAN MOTION

(71) Applicant: The Penn State Research Foundation, University Park, PA (US)

(72) Inventors: Cayce Adam Onks, University Park, PA (US); Ram Mohan Narayanan, University Park, PA (US)

(21) Appl. No.: 19/146,337

(22) PCT Filed: Jan. 8, 2024
(86) PCT No.: PCT/US2024/010642

§ 371 Date: Jul. 8, 2025

Related U.S. Application Data
(60) Provisional application No. 63/437,856, filed on Jan. 9, 2023.

(51) Int. Cl. A61B 5/11; A61B 5/00

(52) U.S. Cl. CPC A61B 5/1126; A61B 5/7225; A61B 5/7257; A61B 5/7267; A61B 5/7271; A61B 5/742; A61B 2503/12

(57) ABSTRACT

Methods, micro-Doppler radar (MDR) systems, and non-transitory computer readable media for evaluating human movement using MDR are disclosed. In some examples, a received radar signal reflected from a human subject in motion is captured via a reception antenna following output via a transmission antenna of a first transmitted radar signal toward the human subject in motion. The first transmitted radar signal is filtered from the received radar signal to yield an analog MDR signal that reflects modulation caused by movement of the human subject. A trained machine learning model is applied to digital micro-Doppler signal (MDS) data converted from the analog MDR signal to generate a result indicative of a likelihood that the human subject is at risk for an injury or other pathological condition. An indication of the result is output for display on a display device to facilitate prediction of the injury or other pathological condition.

This application claims priority to U.S. Provisional Patent Application No. 63/437,856, filed Jan. 9, 2023, which is incorporated by reference herein in its entirety.

This invention was made with Government support under Grant No. W81XWH-22-1-0684 awarded by the United States Army/MRAA. The Government has certain rights in the invention.

### BACKGROUND

Non-combat-related musculoskeletal injuries (MSKI) account for 80% of Service Member injuries, 60% of limited duty days, and 65% of days that US Service Members are unable to deploy. MSKI accounted for $980 billion healthcare dollars in 2014 (5.76% of the annual Gross Domestic Product). MSKI are the greatest medical threat to Service Member health and readiness. In addition, MSKI results in increased military separation, exorbitant medical costs and the development of chronic pain or long-term disability.

The impact of MSKI is not unique to the military. During the 2014-2015 National Basketball Association season, it was estimated that MSKI accounted for a loss of $344 million in player salaries. During the 2019-2020 National Football League season, an estimated $521 million was spent on MSKI. Unlike the general population, both Service Members and athletes represent a younger and more physically fit subpopulation, with MSKI impacting their ability to perform their duties.

However, no effective technologies have been developed to address the burden of MSKI. Primary MSKI risk reduction efforts have shown promise for decreasing MSKI rates; however, current screening methods cannot identify injury risk with sufficient accuracy. While sophisticated assessment tools are available, such as those found at the Center for the Intrepid and National Intrepid Center of Excellence, they are not available outside of these types of dedicated and specialized facilities. Additionally, comprehensive MSKI prediction models currently require extensive expertise and time to administer, which makes use in theater or across the vast military population challenging.

Motion capture (MC) systems that measure three-dimensional joint and body positions are currently used for human movement analysis. Biomechanical analysis of human movement has been extensively studied using passive MC. MC data output consists of three-dimensional coordinates for markers placed on body references such as joint prominences, which are used to illustrate skeletal-like body segments. From there, dynamic analysis of human movement can be performed using common computational methods. Technical developments have improved MC measurement resolution to approximately 1.2-1.5 mm. Compared to a clinical observer, MC systems “see” on a similar level, but have the added advantage of producing relatively accurate marker position data necessary for dynamic analysis.

Despite its utility as a diagnostic tool, MC has significant limitations including: (1) measurement resolution is no better than visual observation; (2) MC measurements often require enhancement using cumbersome secondary measurement methods such as floor-mounted force plates (ground reaction forces) and/or electromyography (muscle activity); (3) MC systems typically require a dedicated lab space designed to minimize external noise sources, effectively rendering MC systems immobile; and (4) MC system costs are in the hundreds of thousands of dollars. Thus, MC is non-portable, expensive, cumbersome to use, and the spatial resolution capabilities are no better than a clinician's vision, and MC therefore suffers from many of the same deficiencies of the current sophisticated assessment tools.

While MSKI is discussed herein as an exemplary injury capable of prediction based on evaluation of human motion, many diseases can be predicted or diagnosed based on an evaluation of human motion. For example, humans with Parkinson's disease often exhibit tremors that upon onset are not visible to the naked eye and are therefore exceedingly difficult to detect. Unfortunately, current human motion analysis methods are ineffective for informing a diagnosis of Parkinson's disease to facilitate early treatment and improved health outcomes. Accordingly, evaluation of human motion is currently not sufficiently effective, portable, inexpensive, granular, or accurate for the prediction or diagnosis of human injuries and other pathologies.

### SUMMARY

In one embodiment, a method for evaluating human movement using micro-Doppler radar (MDR) is disclosed, which is implemented by an MDR system. In some examples, the method includes capturing via a reception antenna a received radar signal reflected from a human subject in motion following output via a transmission antenna of a first transmitted radar signal toward the human subject in motion. The first transmitted radar signal is filtered from the received radar signal to yield an analog MDR signal that reflects modulation caused by movement of the human subject. A trained machine learning model is applied to digital micro-Doppler signal (MDS) data converted from the analog MDR signal to generate a result indicative of a likelihood that the human subject is at risk for an injury or other pathological condition. An indication of the result is then output for display on a display device to facilitate prediction of the injury or other pathological condition.

In one example, the trained machine learning model is configured to employ one or more deep learning algorithms to extract features from the digital MDS data through one or more hierarchical architectures. In another example, the human subject is performing an activity and the method further includes capturing a first MDS data set from a first population of human subjects known to be at risk for the injury or other pathological condition and a second MDS data set from a second populations of healthy human subjects.

In this example, the machine learning model can be trained based on the first and second MDS data sets and deployed when an accuracy threshold is determined to have been exceeded during the training. One or more of a short-term Fourier transform (STFT), a principal component analysis (PCA), or a linear discriminant analysis (LDA) can be applied to one or more of the first MDS data set or the second MDS data set to facilitate the training of the machine learning model.

The trained machine learning model can also be applied to the digital MDS data to determine whether the digital MDS data more likely belongs in the first MDS data set or the second MDS data set in this example. The result includes a binary value generated based on the determination. The trained machine learning model can also be applied to the digital MDS data to generate a score indicative of a likelihood the digital MDS data belongs in the first MDS data set or the second MDS data set, and the result can include the score.

In other examples, a high frequency radar signal is generated via an oscillator. In these examples, the high frequency radar signal is divided via a power divider. A first portion of the divided high frequency radar signal is passed through a power amplifier to generate a first amplified radar signal. The first amplified radar signal is provided to the transmission antenna for output via the transmission antenna as the first transmitted radar signal. In these examples, the received radar signal can be passed through a low noise amplifier to generate a second amplified radar signal. The second amplified radar signal can then be divided via another power divider.

A second portion of the divided high frequency radar signal can also be passed through a ninety-degree hybrid coupler to generate a second transmitted radar signal and a third transmitted radar signal in yet other examples. The third transmitted radar signal is ninety degrees out of phase with respect to the second transmitted radar signal. In these examples, a first portion of the divided second amplified radar signal and the second transmitted radar signal can be passed through a first mixer and a second portion of the divided second amplified radar signal and the third transmitted radar signal can be passed through a second mixer.

In another embodiment, an MDR system is disclosed that includes memory including instructions stored thereon and one or more processors coupled to the memory and configured to execute the stored instructions to perform the method of any of the examples described above. In yet another embodiment, a non-transitory computer readable medium is disclosed that has stored thereon instructions for evaluating human movement using micro-Doppler radar comprising executable code that, when executed by one or more processors, causes the one or more processors to perform the method of any of the examples described above.

The technology described and illustrated herein uses radar to evaluate human movement by applying artificial intelligence methodologies to evaluate radar data, including MDR signals. With this technology, movement characterization and analysis can be made that are not possible using human visual observation, allowing determinations regarding whether humans have or are at risk to sustain or develop a musculoskeletal injury, among other pathologies. The systems and methods of this technology are capable of being used in clinical settings to evaluate human movement and determine if humans are at risk for injury, which in turn allows direction of preventive services to high-risk groups. Thus, this technology may decrease injury incidence, reduce the burden of musculoskeletal injury to society, and inform diagnosis of other human pathologies.

### BRIEF DESCRIPTION OF THE DRAWINGS

FIG. 1 is a block diagram of an exemplary micro-Doppler radar (MDR) system;

FIG. 2 is an exemplary spectrogram representing micro-Doppler signals (MDS) collected from a human in motion;

FIG. 3 illustrates examples of processed short-time Fourier transform (STFT) images for various human movements;

FIGS. 4A-B illustrate predictive accuracy of using MDS to discriminate between micro-motions that result from different human movements;

FIG. 5 illustrates predictive accuracy of using MDS to discriminate between the same movement for humans wearing shoes, a heel lift, and barefoot;

FIG. 6 is a block diagram of an exemplary radar subsystem of the MDR system of FIG. 1;

FIG. 7 is a flow diagram of an exemplary signature processing algorithm implemented by a signature processing module;

FIG. 8 is a flow diagram of another exemplary signature processing algorithm implemented by the signature processing module; and

FIG. 9 is a flowchart of an exemplary method for evaluating human motion using MDR.

### DETAILED DESCRIPTION

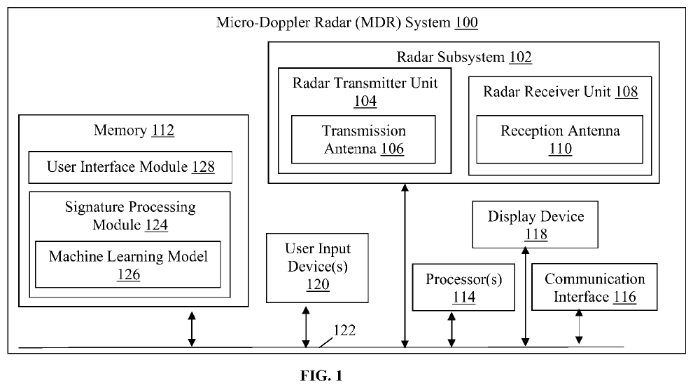

Referring to FIG. 1, an exemplary micro-Doppler radar (MDR) system 100 is illustrated that is portable, has high spatial resolution, is inexpensively produced, and can be readily used with minimal training. The MDR system 100 of this technology is capable of distinguishing slight differences between movement patterns. These minor differences cannot be seen by the human eye, can be used to evaluate differences in movement that have not been visually distinguishable to date, and can be used to find movement patterns that place Service Members, as one exemplary human population, at risk for MSKI, as one exemplary pathology. Accordingly, the technology described and illustrated herein facilitates distinguishing high-risk groups from low-risk groups for MSKI, provides an opportunity for injury prevention, and can facilitate early diagnosis of other pathological conditions.

Analysis of human motion using radar has important applications for both field and garrison operations, search-and-rescue missions, surveillance, and monitoring of hospitalized patients, for example. In the presence of radar illumination, the micro-motions associated with specific human movements create unique and distinct modulations in the received signals, which are referred to herein as micro-Doppler signals (MDS). The MDS associated with these micro-motions produce nonlinear and non-stationary signals that can be characterized using time-frequency domain analysis.

By analyzing the MDS, this technology infers the type of movement being performed. Thus, the disclosed technology can identify MDS that are specific to injury conditions such as anterior cruciate ligament (ACL) tears, stress fractures, or ankle injuries. Once the MDS are known for these injury conditions, this technology can prospectively look for these MDS before a Service Member, for example, experiences an injury-creating an opportunity to direct limited preventive resources to the at-risk Service Member.

The MDR system 100 of this technology includes a radar subsystem 102 with a radar transmitter unit 104 connected to a transmission antenna 106 and a radar receiver unit 108 connected to a reception antenna 110. A radio signal is transmitted into the air using the transmission antenna 106 with a specified directional radiation pattern. The radar signal gets reflected from various objects within the field of view of the transmission antenna 106 in different directions. The part of the reflected radar signal that is in the direction of the radar receiver unit 108, and which lies within the field of view of the reception antenna 110, is captured. The captured part of the radar signal, which contains clues about the target location and movements, is processed by hardware in the radar subsystem 102 and/or software in a memory 112 of the MDR system 100 with specialized processing techniques depending upon the application, as described and illustrated in more detail below.

Radar systems can measure the range, or distance, to the target and the radial velocity (i.e., a target's movement speed either directly towards or away from the radar), depending upon the type of modulation used and the processing approach employed. If a target is stationary and non-moving, the received signal from linear targets, such as most ordinary metallic and non-metallic targets, will be at the same frequency as the transmit frequency (i.e., there will be no associated frequency shift).

When the target is moving, the phenomenon of Doppler effect occurs, which causes a shift in the received frequency compared to the transmitted frequency. The shift in frequency is commonly referred to as the Doppler frequency shift, or simply the Doppler shift. The Doppler shift is positive (i.e., the received frequency is greater than transmitted frequency) for a target approaching a radar observer and it is negative (i.e., the received frequency is lower than transmitted frequency) for a target receding from a radar observer. The amount of Doppler frequency shift (positive or negative) is proportional to the target speed if the transmit frequency is kept constant. In addition, for a target moving at a constant speed, the Doppler shift is proportional to the transmit frequency.

Since most targets are not rigid bodies, there are often other vibrations and rotations in various parts of the target in addition to the platform movement. For example, when a helicopter flies, its blades rotate, and when a person walks, their arms swing naturally, partly in a periodic and partly in a random manner. These micro-scale movements produce additional Doppler shifts referred to as micro-Doppler effects or signatures, which are useful in identifying target features as explained in more detail below.

With this technology, MDS is used to identify human gait patterns and other activities by discerning minute movements and convulsions, which cannot be seen by the naked eye. Standard human biomechanical analysis, such as motion capture systems, have not previously been able to capture movements at this resolution. More specifically, these micro-motions can be captured with this technology to reveal patterns that can facilitate categorization of those at greatest risk for MSKI, or other pathologies, thus identifying where preventative resources can be allocated, among other benefits.

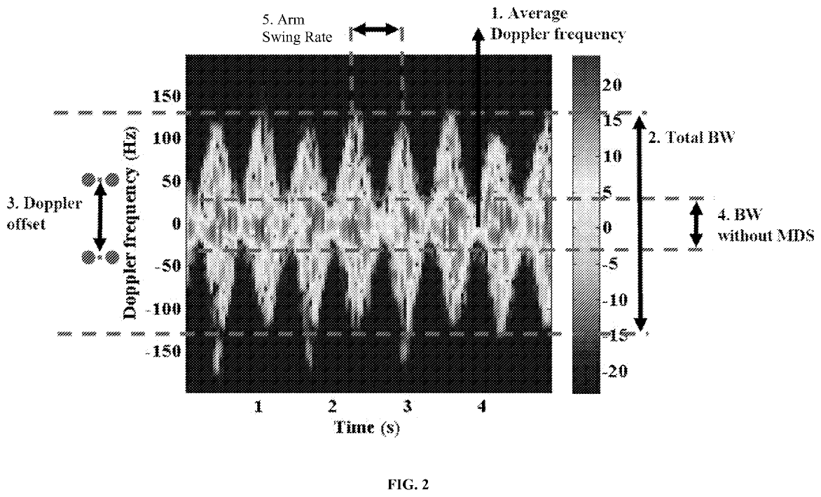

In some examples, the MDS is collected from a subject and the data is represented as a spectrogram (e.g., as shown in FIG. 2). This data can include (1) average Doppler frequency, (2) total Doppler bandwidth, (3) Doppler offset, (4) bandwidth without micro-Doppler, and/or (5) maximum cadence frequency (i.e., arm swing rate in this example). Feature values are useful for classification of human activities using MDS. Labeled characteristics of a subject's movements are then analyzed to understand the most important distinguishing features of the dataset. To do this, the received MDS is analyzed in the time-frequency domain using short-time Fourier transform (STFT). In FIG. 2, frequency changes over time, creating the MDS for a subject as they move in front of the radar. The features of the spectrogram in FIG. 2 that are labeled are used for classifying the movement and to differentiate movement patterns through machine learning processing, as explained in more detail below.

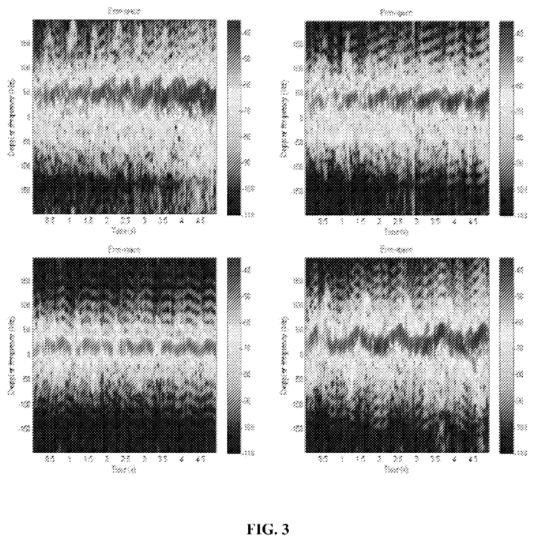

An exemplary implementation of this technology in which data was collected in a free-space environment will now be described. For stationary movements, a subject stood three meters in front of the radar. When performing forward-moving movements, such as gait, the subjects started farther away from the radar at approximately eight meters and moved forward for a duration of five seconds. Examples of the processed STFT images for various movements are shown in FIG. 3, which illustrates differences between the STFT images. With this technology, signatures are mined using deep machine learning algorithms to provide features for classification purposes, as explained in more detail below.

In another example, and with respect to differentiating subtle changes in movement conditions not visible to the human eye, the MDR system 100 of this technology was positioned in front of 12 subjects and the MDS were recorded for each of the activities identified in Table 1:

TABLE 1
LIST OF ACTIVITIES PERFORMED

| | |
|---|---|
| 1. | Walk barefoot towards radar |
| 2. | Jog barefoot towards radar |
| 3. | Jump barefoot facing radar |
| 4. | Jump barefoot turned 90° right of radar |
| 5. | Walk with running/athletic shoes towards radar |
| 6. | Jog with running/athletic shoes toward radar |
| 7. | Jump with running/athletic shoes facing radar |
| 8. | Jump with running/athletic shoes turned 90° right of radar |
| 9. | Walk with a heel lift placed in the right shoe toward radar |
| 10. | Jog with a heel lift placed in the right shoe toward radar |
| 11. | Jump with a heel lift placed in the right shoe facing radar |
| 12. | Jump with a heel lift in the right shoe turned 90° right of radar |

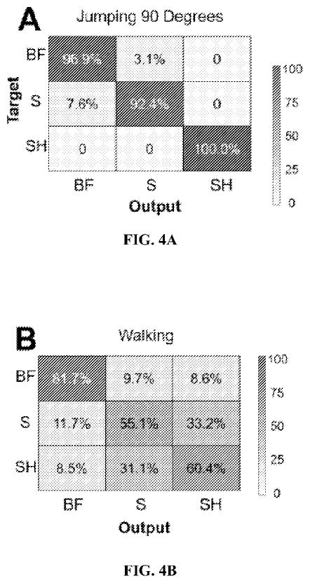

Feature extraction was then performed on the micro-Doppler responses for acquiring unique features commonly used in gait analysis by MDR measurements. A secondary analysis utilized dimensionality reduction through principal component analysis (PCA) and linear discriminant analysis (LDA) to extract unique signatures and perform abnormal gait classification. An in-depth evaluation was performed on the different feature sets using a weighted k-nearest-neighbor (WKNN) and support vector machine (SVM) classifier algorithm. FIG. 4 shows the feasibility of using MDS to discriminate between micro-motions that result from subjects performing tasks either barefoot, with shoes, or with shoes and a 2-cm heel lift.

Referring to FIG. 4A, the SVM classifier algorithm predicted when the subject was jumping barefoot 96.9% of the time and when the subject was jumping with a heel lift 100% of the time in this sample. When the subject was walking, the algorithm successfully predicted who was barefoot 81.7% of the time and if the subject was wearing a heel lift 60.4% of the time (as illustrated in FIG. 4B). From these results, the MDR system 100 determined that jumping was best to discriminate between the three conditions. The algorithm used in this example is also referred to herein as the micro-Doppler signature projection algorithm (mDSPA).

In another example, the MDR system 100 of this technology was used to perform a cross-sectional study to detect known differences in biomechanics among 37 healthy National Collegiate Athletic Association athletes on one college campus, who performed three sets of three squat jumps in front of the MDR system 100. Each set of squat jumps was performed barefoot, with shoes, and with a 2-cm heel lift at both 0 degrees and turned 90 degrees to the reception antenna 110 of the MDR system 100.

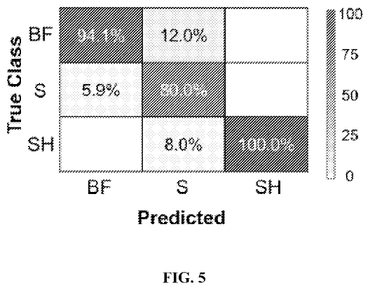

The data was then analyzed using the mDSPA. The overall accuracy of the computer-derived models to predict which subjects were wearing shoes, a heel lift or barefoot was 80%-100%, as illustrated in FIG. 5, for all groups regardless of whether the subject was facing or turned to the right of the reception antenna 110 of the MDR system 100. Notably, the MDR system 100 determined 100% of the time which subject was in a heel lift, which illustrates the sensitivity of the MDR system 100 with respect to predicting subtle differences in movements.

These subtle differences cannot be seen by the human eye and thus the MDR system 100 can be used to evaluate differences in movement that have not been distinguishable to date. Additionally, these subtle differences can be used to find movement patterns that place individuals at risk for MSKI, allowing the MDR system 100 to accurately predict individuals with an elevated risk for MSKI, as explained in more detail below. In turn, limited preventive resources can be utilized to decrease the individual's risk for MSKI.

Referring back to FIG. 1, as explained above, the MDR system 100 includes a radar subsystem 102 whose operating parameters are illustrated in Table 2 in some examples:

TABLE 2

| RF Hardware | | | Signal Processing | | |
|---|---|---|---|---|---|
| Parameters | Units | Value | Parameters | Units | Value |
| Output Power | dBm | 30 | Doppler | Hz | 400 |
| (Antenna Tip) | | | Bandwidth | | |
| Center | GHz | 8.5 | Sampling | kHz | 20 |
| Frequency | | | Frequency | | |
| Power | dB | 25 | PCA Feature | — | 95% |
| Amplifier Gain | | | Variance | | |
| Antenna Type | N/A | Horn | Linear | — | Labels-1 |
| | | | Discriminants | | |
| Antenna | dBi | 20 | Windowing | — | Hamming |
| Directive Gain | | | Function | | |
| Receiver | kHz | 20 | Window Size | samples | 512 |
| Sample Rate | | | | | |
| Data Collect | seconds | 2.5 | STFT overlap | percent | 95 |
| Duration | | | | | |
| Low Pass Filter | kHz | DC - 1 | Detection | dB | −100 |
| Span | | | Threshold | | |

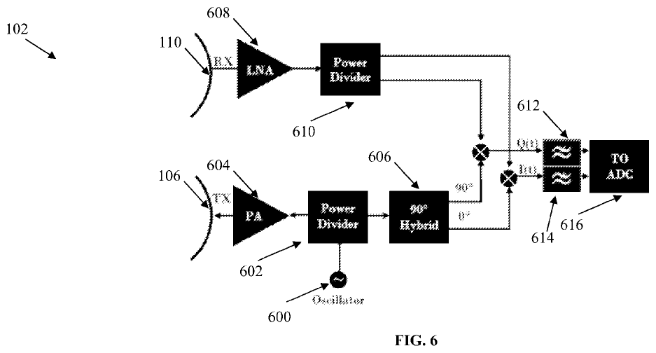

Referring to FIG. 6, an exemplary block diagram is shown, which illustrates an exemplary implementation of the radar subsystem 102 of the MDR system 100, As illustrated in this example, the oscillator 600 generates a high frequency signal that is divided in two directions by the power divider 602. In the direction towards the transmission antenna 106, the divided signal passes through a power amplifier 604 and is then transmitted towards a subject that may be in motion. In the opposite direction, the divided signal passes through a 90-degree hybrid coupler 606 that provides two outputs, one 90 degrees out of phase from the other.

The reception antenna 110 then receives the reflected signal from the subject and the received signal is passed through a low noise amplifier 608 to amplify the signal while adding minimal noise. The power divider 610 again divides that resulting signal into two signals that are passed to the mixers 612, 614 through which the signals output by the 90-degree hybrid coupler 606 are also passed. The mixers 612, 614 perform a comparison between the output signal and the received signal to facilitate stripping out of the high frequency signal to yield a signal that reflects only the modulation caused by the differential movement of the subject, which is then processed by an analog-to-digital converter (ADC) 616 to generate digital MDS that can be processed as explained in more detail below.

Referring back to FIG. 1, in addition to the radar subsystem 102, the MDR system 100 includes processor(s) 114, the memory 112, a communication interface 116, a display device 118, and user input device(s) 120, which are coupled together by a bus 122, although the MDR system 100 can include other types or numbers of elements in other configurations. The processor(s) 114 of the MDR system 100 may execute programmed instructions stored in the memory 112 of the MDR system 100 for any number of the functions described and illustrated herein. The processor(s) 114 may include one or more general purpose processors with one or more processing cores, one or more central processing units (CPUs), and/or one or more graphics processing units (GPUs), for example, although other types of processor(s) can also be used.

The memory 112 stores these programmed instructions for one or more aspects of the present technology as described and illustrated herein, although some or all the programmed instructions could be stored elsewhere. A variety of different types of memory storage devices, such as random access memory (RAM), read only memory (ROM), hard disk, solid state drives, flash memory, or other computer readable medium which is read from and written to by a magnetic, optical, or other reading and writing system that is coupled to the processor(s) 114, can be used for the memory 112.

Accordingly, the memory 112 can store applications that can include computer executable instructions that, when executed by the processor(s) 114, cause the MDR system 100 to perform actions, such as to transmit, receive, or otherwise process network messages and requests, for example, and to perform other actions described and illustrated below. The application(s) can be implemented as components of other applications, operating system extensions, and/or plugins, for example.

In this example, the memory 112 includes a signature processing module 124 with a machine learning model 126 and a user interface module 128, although other modules and/or applications can also be provided in other examples. The signature processing module 124 is illustrated in software in FIG. 1, although one or more functions provided by the signature processing module 124 can be implemented in hardware within the radar subsystem 102 in other examples. The signature processing module 124 in this example is configured to extract features from time-frequency and/or cadence-frequency diagrams (e.g., the time-frequency representations of the MDS generated by the radar subsystem 102). Time-frequency signatures implement the STFT for analyzing MDS signatures in some examples.

After constructing signatures for each activity measured by the MDR system 100, further processing is performed by the signature processing module 124 on the signatures to extract unique information. A variety of methods could be employed by the MDR system 100 for extracting features embedded in the signatures, including methods handcrafted from the raw or simulated data and other methods that are extracted through machine learning techniques, such as dimensionality reduction. Thus, in some examples, the MDR system 100 is configured to execute a dimensionality reduction technique in the classification of human gait, for example, via MDR.

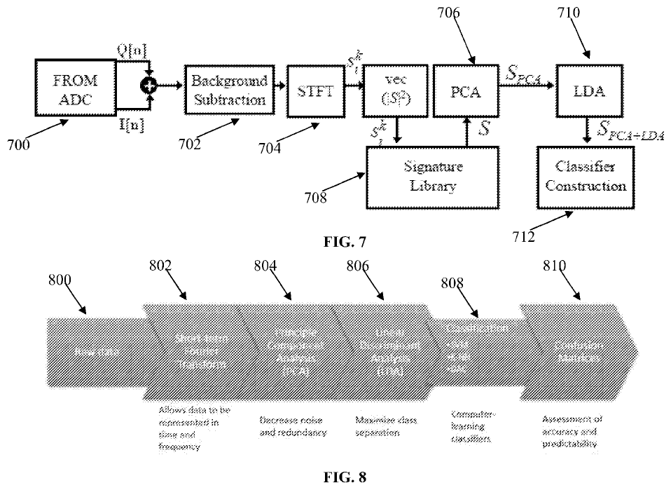

Referring to FIG. 7, a flow diagram of an exemplary signature processing algorithm implemented by the signature processing module 124 is illustrated. In step 700 in this example, the signature processing module 124 receives the digital signals converted by the ADC 616 of the radar subsystem 102, for example. The signature processing module 124 performs a background subtraction on the received digital signals in step 702 followed by a STFT in step 704 to put the digital signals in a time and frequency domain.

The signature processing module 124 then implements a PCA on the resulting digital signatures in step 706 to eliminate noise and redundancy, for example. The PCA uses a signature library 708 of known activity signatures in this example. The signature processing module 124 then implements an LDA in step 710 and constructs classifiers (e.g., machine learning classifiers) to generate the machine learning model 126 in step 712. The signature processing module 124 can then optionally utilize confusion matrices to assess the accuracy of the machine learning model 126.

Referring to FIG. 8, a flow diagram of another exemplary signature processing algorithm implemented by the signature processing module 124 is illustrated. In this example, the signature processing module 124 receives raw data in step 800, such as digital signals from the radar subsystem 102, for example. The signature processing module 124 implements an STFT in step 802 to represent the digital signal data in time and frequency domains. The PCA and LDA are then performed by the signature processing module 124 in step 804 and 806, respectively, to decrease noise and redundancy and maximize class separation, respectively.

The signature processing module 124 then implements machine learning classification in step 808, which can include using SVM and/or k-nearest neighbor (KNN) classification algorithms. Confusion matrices are then utilized by the signature processing module 124 in step 810 to assess accuracy and predictability of the machine learning model 126 associated with the classification.

Additionally, in some examples the machine learning model 126 is configured to employ a deep learning algorithm to extract high-level deep features automatically through hierarchical architectures to enhance classification accuracies for MDS data. Exemplary deep learning approaches include dictionary learning, deep convolutional neural networks, tower convolutional neural networks, temporal convolutional, deep neural networks, and joint domain and semantic transfer learning. In dictionary learning, the dictionary is learned for each type of gait or other activity by using the K-SVD algorithm on cadence-velocity diagrams of gait MDS, and by exploiting their sparse properties, the K-SVD algorithm can be used to decompose these into potential features.

With more layers in the architecture compared to convolutional neural networks, deep convolutional neural networks are specially designed to classify images (such as STFT images) and include the VCG-16 and ResNet-50 architectures. Tower convolutional neural networks employ parallel input layers with individual color-channel images sent as inputs to the machine learning model 126, wherein all the unique signature features from each channel are concatenated to have better and robust feature representation to the machine learning model 126.

Temporal convolutional deep neural networks work with a convolution architecture design, are characterized by causality and an output sequence of constant length, and are particularly suitable to the gait recognition because, in this context, the causal relationships of the gait signal evolution can be learned. Additionally, joint domain and semantic transfer learning combines unsupervised domain adaptation and supervised semantic transfer. Employing a sparsely labeled dataset to train the human activity recognition model alleviates the need of labeling a substantial number of radar signals.

Referring back to FIG. 1, the user interface module 128 of the memory 112 of the MDR system 100 is configured to present graphical user interface(s) (GUI(s)) that present the result of the application of the trained machine learning model 126 to current MDS data. Accordingly, the machine learning model 126 can be applied by the MDR system 100 to currently obtained MDS data (e.g., the signal data output by the radar subsystem 102) to place the MDS data into a class indicative of whether the current MDS data for a subject reflects an injury risk or pathology, for example. The result can be binary or a confidence score reflective of the likelihood that a current subject is at risk for MSKI, for example.

The GUI(s) with the result of the application of the machine learning model 126 to current MDS data can then be output to the display device 118 to provide real-time feedback for an assessed subject, as explained in more detail below. In other examples, the user interface module 128 can further be configured to provide forms or questionnaires to obtain demographic and other information regarding subjects that can inform the classification of respective MDS data for the subjects, and other types of GUIs can also be generated and provided by the user interface module 128.

The optional communication interface 116 of the MDR system 100 operatively couples and communicates between the MDR system 100, user devices, and/or backend databases or servers, which are coupled together by communication network(s). For example, the MDR system 100 can send collected MDS data to a server implementing the signature processing module 124 and receive a result of that implementation via communication network(s) in an SaaS deployment of this technology.

In another example, the machine learning model 126 is downloaded or installed on the MDR system 100 from a server via communication network(s) after being trained on the server or another device with increased processing capabilities, for example. Other topologies can also be used in other examples. Thus, the communication network(s) can employ any suitable interface mechanisms and network communication technologies including, for example, Ethernet-based Packet Data Networks (PDNs). Additionally, the communication interface 116 can include a Bluetooth radio, near-field communication (NFC) interface, Wi-Fi transceiver, or any other type of wired or wireless communication interface.

The display device 118 of the MDR system 100 can be a display screen, touchscreen, or any other device capable of communicating with the user interface module 128 to display GUIs, for example. The user input device(s) 120 of the MDR system 100 can include a touchscreen, keyboard, or mouse, for example. Accordingly, the MDR system 100 can be a laptop, tablet, or other handheld computing device that is portable and low cost to produce.

It is to be understood that the systems of the examples described herein are for exemplary purposes, as many variations of the specific hardware and software used to implement the examples are possible, as will be appreciated by those skilled in the relevant art(s). The examples of this technology may also be embodied as one or more non-transitory computer readable media having instructions stored thereon, such as in the memory 112 of the MDR system 100, for one or more aspects of the present technology, as described and illustrated by way of the examples herein. The instructions in some examples include executable code that, when executed by one or more processors, such as the processor(s) 114 of the MDR system 100, cause the processors to carry out steps necessary to implement the methods of the examples of this technology that will now be described and illustrated herein.

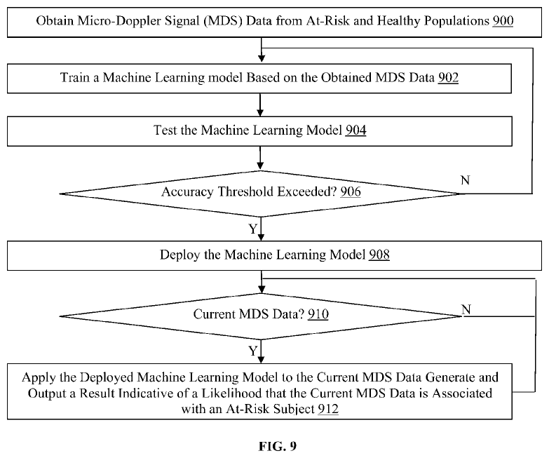

Referring now to FIG. 9, a flowchart of an exemplary method for evaluating human motion using MDR is illustrated. In step 900 in this example, the MDR system 100 obtains MDS signal data from at-risk and healthy populations of subjects. In one example, the at-risk population can have a known susceptibility to future injury. For example, subjects with a prior ACL surgery are susceptible to a future tear or other injury and therefore are at-risk. In other examples, the MDR system 100 can generate a database of MDS data for a population and subsequently tag the MDS data associated with subjects that exhibited an injury or other pathology subsequent to capture of the associated MDS data and generation of the database.

The MDS data can be obtained by the radar subsystem 102 as explained in more detail above and the subjects of the at-risk and healthy population can perform any number of movements of physical activities during the MDS data capture. For example, the subjects can be instructed to walk away and then towards the stationary MDR system 100 to facilitate the capture of MDS data relating to the gait of the subjects.

In step 902, the MDR system 100 trains the machine learning model 126 based on the obtained MDS data and, optionally, other risk factors such as sex, fatigue, pain, and/or body mass index (BMI), for example. The machine learning model 126 can be trained as explained in more detail above, optionally after a threshold or significant amount of MDS data is obtained from the at-risk and healthy populations in step 900. Also optionally, a subset of the obtained MDS data is used to train the machine learning model 126 to distinguish between MDS data captured from a subject in the at-risk population from other MDS data captured from another subject in the healthy population.

In step 904, the MDR system 100 tests the machine learning model. In some examples, the machine learning model 126 is evaluated via a supervised learning process using another subset of the MDS data obtained in step 900 that was not used in the training in step 902. Optionally, the machine learning model 126 can be tested using a cross-validation methodology, although any other testing method(s) can also be used in other examples.

In step 906, the MDR system 100 determines whether an accuracy threshold is exceeded. The accuracy threshold can be configurable and can be analyzed based on results of the testing in step 904. If the MDR system 100 determines that the accuracy threshold has not been exceeded, then the No branch is taken in this example and the MDR system 100 proceeds back to step 902 and continues training the machine learning model 126 or begins training a new machine learning model. However, if the MDR system 100 determines in step 906 that the accuracy threshold has been exceeded, then the Yes branch is taken from step 906 to step 908.

In step 908, the MDR system 100 deploys the machine learning model 126, In some examples, the machine learning model 126 can be deployed by a remote server to any number of MDR systems 100 that may be utilized in clinical environments, for example. The remote server optionally performs one of more of steps 900-908 in some examples, with the MDS signal data being obtained from any number of MDR systems 100 in step 900. In these examples, the signature processing module 124 of the various MDR systems 100 to which the machine learning model 126 is deployed may only be configured to performs steps 910-914 described and illustrated in detail below.

In step 910, the MDR system 100 determines whether current MDS data is obtained from a current assessment of a subject. If the MDR system 100 determines that current MDS data has not been obtained, then the No branch is taken back to step 910 and the MDR system 100 effectively waits until it is utilized to assess a current subject. However, if the MDR system 100 determines that current MDS data has been obtained from a subject, then the Yes branch is taken to step 912.

In step 912, the MDR system 100 applies the deployed machine learning model 126 to the current MDS data to generate and output a result indicative of a likelihood that the current MDS data is associated with an at-risk subject or a healthy subject. In some examples, the generated result is a binary value indicative of whether the MDS data corresponds to a subject in an at-risk population or a healthy population. In another example, the generated result is a score of confidence value that reflects a likelihood that the MDS data corresponds to a subject in the at-risk population or the healthy population. Other types of results can be generated and/or output in other examples.

Accordingly, this technology leverages machine learning to identify patients with pathologic movement patterns that can lead to MSKI more effectively, efficiently, and in a way that neither MC systems nor experienced clinicians can currently detect. Advantageously, this technology has a resolution that is superior to that of MC or the human eye. Identifying subjects that are at highest risk for MSKI can provide an opportunity to direct established preventive resources to this high-risk group.

The MDR system 100 of the disclosed technology can be produced in a small tablet computing device type form factor at a relatively low cost, allowing it to be implemented broadly in various geographic and physical locations (e.g., Service Members in garrison and in the field). The MDR system 100 of the examples described and illustrated herein also can be deployed and used with minimal training requirements.

Thus, the MDR system 100 of this technology provides for more cost-efficient, portable, and accurate detestation of movement deficiencies common to many disabling conditions. Early detection can significantly improve patient outcomes, Service Member readiness, and prevent chronic pain and disability.

Having thus described the basic concept of the invention, it will be rather apparent to those skilled in the art that the foregoing detailed disclosure is intended to be presented by way of example only and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations, therefore, is not intended to limit the claimed processes to any order except as may be specified in the claims. Accordingly, the invention is limited only by the following claims and equivalents thereto.

1-13. (canceled)

14. A method for evaluating human movement using micro-Doppler radar (MDR), the method implemented by an MDR system and comprising:
capturing via a reception antenna a received radar signal reflected from a human subject in motion following output via a transmission antenna of a first transmitted radar signal toward the human subject in motion;
filtering the first transmitted radar signal from the received radar signal to yield an analog MDR signal that reflects modulation caused by movement of the human subject;
applying a trained machine learning model to digital micro-Doppler signal (MDS) data converted from the analog MDR signal to generate a result indicative of a likelihood that the human subject is at risk for an injury or other pathological condition; and
outputting an indication of the result for display on a display device to facilitate prediction of the injury or other pathological condition.

15. The method of claim 14, wherein the human subject is performing an activity during the capture of the received radar signal and the method further comprises capturing a first MDS data set from a first population of human subjects known to be at risk for the injury or other pathological condition and a second MDS data set from a second populations of human subjects healthy with respect to the injury or other pathological condition.

16. The method of claim 15, further comprising training the machine learning model based on the first and second MDS data sets and deploying the trained machine learning model when an accuracy threshold is determined to have been exceeded during the training.

17. The method of claim 15, further comprising applying the trained machine learning model to the digital MDS data to determine whether the digital MDS data more likely belongs in the first MDS data set or the second MDS data set, wherein the result comprises a binary value generated based on the determination.

18. The method of claim 15, further comprising applying the trained machine learning model to the digital MDS data to generate a score indicative of a likelihood the digital MDS data belongs in the first MDS data set or the second MDS data set, wherein the result comprises the score.

19. The method of claim 16, further comprising applying one or more of a short-term Fourier transform (STFT), a principal component analysis (PCA), or a linear discriminant analysis (LDA) to one or more of the first MDS data set or the second MDS data set to facilitate the training of the machine learning model.

20. The method of claim 14, wherein the trained machine learning model is configured to employ one or more deep learning algorithms to extract features from the digital MDS data through one or more hierarchical architectures.

21. A micro-Doppler radar (MDR) system, comprising a reception antenna, a transmission antenna, memory having instructions stored thereon for evaluating human movement using MDR, and one or more processors coupled to the memory and configured to execute the stored instructions to:
capture via the reception antenna a received radar signal reflected from a human subject in motion following output via the transmission antenna of a first transmitted radar signal toward the human subject in motion;
filter the first transmitted radar signal from the received radar signal to yield an analog MDR signal that reflects modulation caused by movement of the human subject;
apply a trained machine learning model to digital micro-Doppler signal (MDS) data converted from the analog MDR signal to generate a result indicative of a likelihood that the human subject is at risk for an injury or other pathological condition; and
output an indication of the result for display on a display device to facilitate prediction of the injury or other pathological condition.

22. The MDR system of claim 21, further comprising an oscillator, a first power divider, and a power amplifier, wherein the one or more processors are further configured to execute the stored instructions to:
generate via the oscillator a high frequency radar signal;
divide via the first power divider the high frequency radar signal;
pass a first portion of the divided high frequency radar signal through the power amplifier to generate a first amplified radar signal; and
provide the first amplified radar signal to the transmission antenna for output via the transmission antenna as the first transmitted radar signal.

23. The MDR system of claim 22, further comprising a low noise amplifier and a second power divider, wherein the one or more processors are further configured to execute the stored instructions to:
pass the received radar signal through the low noise amplifier to generate a second amplified radar signal; and
divide via the second power divider the second amplified radar signal.

24. The MDR system of claim 23, further comprising a ninety-degree hybrid coupler, wherein the one or more processors are further configured to execute the stored instructions to pass a second portion of the divided high frequency radar signal though the ninety-degree hybrid coupler to generate a second transmitted radar signal and a third transmitted radar signal, wherein the third transmitted radar signal is ninety degrees out of phase with respect to the second transmitted radar signal.

25. The MDR system of claim 24, further comprising a first mixer and a second mixer, wherein the one or more processors are further configured to execute the stored instructions to pass a first portion of the divided second amplified radar signal and the second transmitted radar signal through the first mixer and a second portion of the divided second amplified radar signal and the third transmitted radar signal through the second mixer.

26. A non-transitory computer readable medium having stored thereon instructions for evaluating human movement using micro-Doppler radar (MDR) comprising executable code that, when executed by one or more processors, causes the one or more processors to:
train a machine learning model based on a first micro-Doppler signal (MDS) data set captured from a first population of human subjects known to be at risk for an injury or other pathological condition and a second MDS data set captured from a second population of human subjects healthy with respect to the injury or other pathological condition;
capture via a reception antenna a received radar signal reflected from a human subject in motion following output via a transmission antenna of a first transmitted radar signal toward the human subject in motion;
filter the first transmitted radar signal from the received radar signal to yield an analog MDR signal that reflects modulation caused by movement of the human subject;
apply a trained machine learning model to digital MDS data converted from the analog MDR signal to generate a result indicative of a likelihood that the human subject is at risk for the injury or other pathological condition; and
output an indication of the result for display on a display device to facilitate prediction of the injury or other pathological condition.

27. The non-transitory computer readable medium of claim 26, wherein the executable code, when executed by the one or more processors, further causes the one or more processors to apply the trained machine learning model to the digital MDS data to determine whether the digital MDS data more likely belongs in the first MDS data set or the second MDS data set, wherein the result comprises a binary value generated based on the determination.

28. The non-transitory computer readable medium of claim 26, wherein the executable code, when executed by the one or more processors, further causes the one or more processors to apply the trained machine learning model to the digital MDS data to generate a score indicative of a likelihood the digital MDS data belongs in the first MDS data set or the second MDS data set, wherein the result comprises the score.

29. The non-transitory computer readable medium of claim 28, wherein the executable code, when executed by the one or more processors, further causes the one or more processors to apply one or more of a short-term Fourier transform (STFT), a principal component analysis (PCA), or a linear discriminant analysis (LDA) to one or more of the first MDS data set or the second MDS data set to facilitate the training of the machine learning model.

30. The non-transitory computer readable medium of claim 26, wherein the trained machine learning model is configured to employ one or more deep learning algorithms to extract features from the digital MDS data through one or more hierarchical architectures.

31. The non-transitory computer readable medium of claim 26, wherein the executable code, when executed by the one or more processors, further causes the one or more processors to:
generate via an oscillator a high frequency radar signal;
divide via a power divider the high frequency radar signal;
pass a first portion of the divided high frequency radar signal through a power amplifier to generate a first amplified radar signal;
provide the first amplified radar signal to the transmission antenna for output via the transmission antenna as the first transmitted radar signal;
pass the received radar signal through a low noise amplifier to generate a second amplified radar signal; and
divide via another power divider the second amplified radar signal.

32. The non-transitory computer readable medium of claim 31, wherein the executable code, when executed by the one or more processors, further causes the one or more processors to pass a second portion of the divided high frequency radar signal though a ninety-degree hybrid coupler to generate a second transmitted radar signal and a third transmitted radar signal, wherein the third transmitted radar signal is ninety degrees out of phase with respect to the second transmitted radar signal.

33. The non-transitory computer readable medium of claim 32, wherein the executable code, when executed by the one or more processors, further causes the one or more processors to pass a first portion of the divided second amplified radar signal and the second transmitted radar signal through a first mixer and a second portion of the divided second amplified radar signal and the third transmitted radar signal through a second mixer.
